(19) United States
(12) Patent Application Publication
(10) Pub. No.: US 2010/0028849 A1
(43) Pub. Date: Feb. 4, 2010

(54) NOVEL PROCESS FOR DEVITALIZED/ACELLULAR TISSUE FOR TRANSPLANTATION

(76) Inventors: Nancy Jane Shelby, Bozeman, MT (US); Kristin Leigh Fitzpatrick, Bozeman, MT (US); Darrel Lee Holmes, Bozeman, MT (US)

Correspondence Address: KINNEY & LANGE, P.A., THE KINNEY & LANGE BUILDING, 312 SOUTH THIRD STREET, MINNEAPOLIS, MN, 55415-1002, US

(21) Appl. No.: 12/311,964

(22) PCT Filed: Oct. 18, 2007
(86) PCT No.: PCT/US2007/022225

§ 371 Date: Apr. 20, 2009

Related U.S. Application Data
(60) Provisional application No. 60/852,915, filed on Oct. 18, 2006.

(51) Int. Cl. A01N 1/02; C12N 9/99

(52) U.S. Cl. 435/1.2; 435/184; 435/284.1

(57) ABSTRACT

A method for processing tissue to produce a devitalized acellular matrix for transplantation comprises soaking the tissue in a first processing solution having a pH below 7 to reduce protease activity, periodically infusing ozone into the first processing solution to devitalize the tissue and reduce bioburden, and soaking the tissue in a second processing solution to remove cellular debris. The present invention also includes a system for processing the tissue. The devitalized acellular matrix acts as a scaffold for cellular ingrowth when transplanted into a recipient to form new tissue.

### BACKGROUND

The present invention relates to a devitalized acellular matrix for transplantation. More specifically, the present invention relates to a method and system for processing tissue in a solution having a pH below 7 and exposing the tissue to ozone to form a devitalized acellular matrix for transplantation.

Skin is comprised of two primary layers: the epidermis and the dermis. The dermis is the underlying layer, and is the thickest. The dermis provides the structure for the skin organ with a robust extracellular matrix, and has an extensive vascular system that provides the epidermis with nutrients. It also regulates body temperature.

Acellular dermis has been utilized at various times since the early 1900s in wound healing applications to augment soft tissue repair. (Medawar P B. “The Storage of Living Skin.” Proc R Soc Med. 47(1) (1954): 62-4). Cryopreserved cadaver skin (with probably no viable cells) was used early on to investigate the possibility of using acellular skin to support cell growth and heal wounds. In the mid-1970s, acellular dermis was seeded in vitro with fibroblasts and transplanted into athymic mice where it was established as a useful wound healing approach. (Moserova J, et al. “Experimental Comparison of Different Skin Substitutes.” Zentralbl. Chir. 106 (1981): 1194). Clinically acellular dermis has been used extensively as a matrix without cell seeding, and has been used since 1995 to treat full thickness burns and other conditions. (Wainwright D. J. “Use of an Acellular Allograft Dermal Matrix (Alloderm) in the Management of Full-Thickness Burns.” Burns 21 (1995): 243). Past studies have shown that acellular dermis is able to support host fibroblast infiltration, neovascularization and reepithelialization with minimal inflammatory response. (Hodde J. Naturally Occurring Scaffolds For Soft Tissue Repair and Regeneration.” Tissue Engineering 8 (2002): 295). When transplanted into a patient, an optimal acellular dermis will be incorporated into the surrounding tissue, revascularized and repopulated with the patient's own cells (or cells seeded prior to transplantation), and will become functional, normal tissue over time.

Critical to the preparation of acellular dermis is the devitalization of the cells in the donor tissue, followed by successful removal of cellular debris, to reduce the antigenicity of the tissue, preventing rejection and inflammation, which would delay the wound healing process. Cadaveric skin is harvested from donors, placed into a nutrient media at 4° C., and sent for processing. It has been shown that for short periods of time, storage of cadaveric skin tissue at 4° C. in a nutrient media maintains significant viability of skin cells, in part due to the lower temperature, which decreases metabolic activity. (Fitzpatrick K., et al. “Enhanced Viability of Stored Human Skin Using Glycosaminoglycan Enriched Media.” In Preparation (2007)). However, during storage, the cells are often stressed due to ischemia, initiating many stress related biochemical pathways that may negatively affect the extracellular matrix. The cellular production of proteases, including matrix metalloproteinase-1 and others in this family, is increased during ischemia and may begin collagen degradation processes prior to the death of cells.

A protease's activity is strongly dependent on the pH of its surroundings (Schultz G. S., et al. “Inflammatory Proteases in Chronic Otitis Externa.” Larvngoscope 115 (2005): 651). In studies of wound microenvironments, it has been found that open wounds tend to have a neutral or alkaline pH, predominantly in the range of 6.5-8.5. (Dissemond et al. “pH Values in Chronic Wounds.” Hautarzt 54(10) (2003): 959-65). Since chronic wounds can be described as having permanently elevated protease levels resulting in a prolonged inflammatory state, one strategy to promote healing may be to decrease the proteolytic activity to the normal levels observed in acute wounds (post-48 hours) by use of a pH modulator. A weakly acidic environment may promote healing in open wounds by inhibiting the action of proteases. (Leveen et al. “Chemical Acidification of Wounds.” Ann Surg. 178(6) (1973): 745-53).

For example, lowering wound pH to around 5 dramatically slows down the activity of harmful proteases, which can break down the newly formed matrix and also cause prolonged inflammation. (Greener et al. “Proteases and pH in Chronic Wounds.” J Wound Care February; 14(2) (2005): 59-61). In addition, lowering the pH from 8 to 4 can reduce protease activity by 80%. (Schultz et al., 2005). Greener et al. states that: “wound pH must be greater than 4 for healing activity to take place and less than 7 to avoid degradation of the newly formed matrix.” These investigators demonstrated that the pH-dependent activity profiles of four proteases important in wound healing, cathepsin G, elastase, plasmin, and MMP-2, showed peak enzyme levels, where the protein is broken down more rapidly than at other pH values. The group of proteases observed in the Greener et al. study had a similar mechanism of action and revealed similar pH profiles when levels of degradation of a gelatin film were examined using laser imaging and staining.

Currently, various cell extraction methods are used to create acellular tissues, using chemical, mechanical and enzymatic approaches. All of the published approaches appear to have a lengthy period of tissue ischemia before cessation of cellular metabolism, through tissue devitalization with cellular rupture or cryopreservation. However, there is no published process that begins with devitalization of the cells in tissue, without cell disruption, as an early step in the process

Ozone is a gas known to have kill activity for organisms during exposure of cells, bacteria and viruses. It is utilized extensively in water purification and the kinetics of exposure to kill for many organisms are known. “Chemical disinfection by ozone can be achieved by bringing water in contact with gaseous ozone for a certain period of time. The kinetics of the deactivation of pathogenic microorganisms (disinfection) is comparable to a chemical reaction. The most commonly used model to describe water disinfection by ozone is the Chick-Watson law. This law can be mathematically represented as follows: k=Cⁿ·t; k=reaction-constant, dependent on the type of microorganism and the disinfectant; C=disinfectant concentration; t=contact time, period of time that the disinfectant is in contact with water; n=constant. In most cases n equals 1, causing the deactivation of bacteria to become a first-order reaction. When the n constant (nearly) equals 1, Watson's law can be approached as: k=C·t. During disinfection, this Ct-value is used. This value is a multiplication of the disinfectant concentration (C) in mg/L and contact time (t) in minutes, which is needed to deactivate a microorganism. Various levels of deactivation can be achieved. This is often expressed as a log reduction: 1 log reduction=90% deactivation; 2 log reduction=99% deactivation; 3 log reduction=99.9% deactivation; 4 log reduction=99.99% deactivation. Much research has been conducted on Ct-values for various types of microorganisms and for various disinfectants. Data on Ct-values in literary sources may differ. While comparing disinfectants, the Ct-value must always be associated with the log reduction. Apart from concentration and time there are other factors that influence this Ct-value. Examples are pH value, sunlight, water temperature, mixture of water and the disinfectant, and contact chamber design.” (“Kinetics of Ozone Disinfection.” Retrieved Oct. 18, 2006 from http://www.lenntech.com/ozone/ozone-disinfection-kinetics.htm).

There is a need in the art for a method and system for producing a devitalized acellular matrix while reducing tissue ischemia, protecting the integrity of the matrix and reducing bioburden.

### SUMMARY

The present invention is a method for processing tissue to produce a devitalized acellular matrix for transplantation. The method comprises soaking the tissue in a first processing solution having a pH below 7 to reduce protease activity, periodically infusing ozone into the first processing solution to devitalize the tissue and reduce bioburden, and soaking the tissue in a second processing solution to remove cellular debris. The present invention also includes a system for processing the tissue. The devitalized acellular matrix acts as a scaffold for cellular ingrowth when transplanted into a recipient to form new tissue.

### BRIEF DESCRIPTION OF THE DRAWINGS

FIG. 1 is a flow diagram illustrating a method for processing tissue to produce a devitalized acellular matrix.

FIG. 2 is a diagram of a system for processing tissue to produce a devitalized acellular matrix.

FIGS. 3A-3D illustrate a skin fixturing and processing chamber in detail.

FIG. 4 is a chart illustrating the effect of ozone on human skin.

FIG. 5 is a chart illustrating the reduction in residual DNA on human skin when it is subjected to a decellularization process.

### DETAILED DESCRIPTION

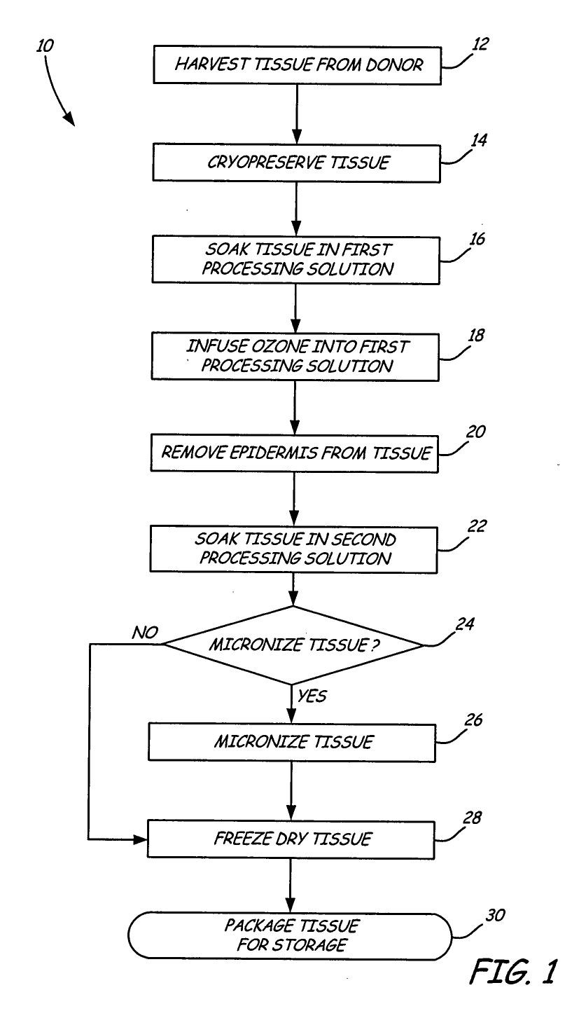

FIG. 1 is a flow diagram for method 10, which is an exemplary embodiment for processing mammalian soft tissue to produce a devitalized acellular matrix. As described above, acellular tissues act as scaffolds for cellular ingrowth when transplanted into a recipient. Over days and weeks, the recipient's body repopulates the matrix and forms new tissue. Acellular dermis is one of the most frequently transplanted acellular tissues. However, the invention is not so limited and other types of soft tissue may be processed, such as blood vessel, nerve, muscle, tendon, pericardium, dura, fascia lata, placenta, and omentum tissue.

Method 10 includes steps 12-30 and initially involves harvesting cadaveric tissue from a donor (step 12). The tissue may be placed in an isotonic nutrient media, such as Roswell Park Memorial Institute media (RPMI 1640) manufactured by Invitrogen Corporation in Carlsbad, Calif. Antibiotics may be added to the RPMI 1640 solution and the tissue is stored at 4° C. so it can be shipped to a tissue processing facility. Upon receipt of the tissue at the tissue processing facility, the skin may be stored in RPMI 1640 with antibiotics at 4° C. for a short period of time. The storage solution (RPMI 1640 or similar nutrient media) maintains the integrity of the tissue and viability of the cells through buffers and nutrients, and tissue in this solution has been shown to maintain viability of the cells at 4° C. for a short period of time, thereby inhibiting degradation of cells and extracellular matrix. The tissue is then cryopreserved until processing is continued (step 14). (However, the present invention is not so limited, and cryopreservation is not necessary if the tissue is processed very soon after arriving at the tissue processing facility.)

In order to continue processing the tissue, the cryopreserved tissue is thawed. The tissue is then placed in a first processing solution for soaking (step 16). The first processing solution has a pH below 7 and acts to reduce protease activity within the tissue. In an exemplary embodiment, the first processing solution has a pH of 5.0 to 6.8. The first processing solution may also contain protease inhibitors and other agents, such as antibiotics, to protect the matrix. Ozone is then infused into the first processing solution to devitalize the tissue (step 18). The ozone acts to kill the cellular components of the tissue while the lower pH of the solution inhibits the activity of proteases that otherwise may degrade the extracellular matrix, including collagen. The tissue is exposed to the ozone for a duration validated to ensure cessation of metabolic activity of the viable cells within the tissue due to death of the cells. Stopping all metabolic processes as early as possible in the devitalization process also optimizes the quality of the matrix. A high quality matrix has been shown to have a longer duration in the host, which allows for optimal tissue formation. In an exemplary embodiment, ozone was infused into the first processing solution for a time period ranging from about 1 minute to about 3 hours resulting in the first processing solution having an ozone concentration of about 0.5 parts per million (ppm) to about 100 ppm. In addition, step 18 may be performed with or without agitation of the tissue.

Steps 16 and 18 of method 10 are carried out in an aseptic environment as the tissue is being devitalized and prepared for decellularization. The aseptic environment and all equipment may be chemically decontaminated following procedures known in the art. Bioburden cultures are performed at the time of harvest, receipt into the facility, and after every procedure during the processing. Reduction of bioburden, as determined by microbiologic sampling, by ozone treatment will be monitored, with anticipation of greater than 6 log reduction in bacterial load with described treatment.

The tissue is then placed into 1 M NaCl solution for up to 24 hours for the purpose of removing the epidermis from the dermis (step 20). Other agents may include trypsin or dispase as manufactured by Sigma-Aldrich Corp. of St. Louis, Mo. The tissue is then thoroughly rinsed.

Once the tissue is thoroughly rinsed, the tissue is placed into a second processing solution to remove cellular components (step 22). The second processing solution may contain detergents, such as polysorbate 20 or Triton® X-100, and endonucleases, such as Benzonase® manufactured by EMD Chemicals Inc. of Gibbstown, N.J. In addition, the second processing solution may also contain antibiotics and have a pH of less than 7.0. The tissue is soaked in the second processing solution for a period of time sufficient to remove all cellular and other detached debris, leaving only the collagen structural matrix and associated proteins. Ozone exposure may again be applied to the tissue to further reduce bioburden and mildly crosslink the tissue. The tissue is then thoroughly rinsed and may also be cryopreserved.

The tissue is then prepared for freeze-drying or for micronization and subsequent freeze drying. The tissue is packaged and sealed in a type of package that will allow water vapor and gases (including ozone) to pass through while not allowing bacteria, dust, etc. contact with the tissue. One such suitable packaging material is Tyvek™.

A decision is then made whether to micronize the tissue or proceed directly to freeze drying (step 24). Whether the tissue is micronized before freeze drying depends upon how the devitalized acellular matrix will be utilized. For example, if the devitalized acellular matrix is intended for use on a large area, it may be freeze dried in sheets and micronization is not necessary. However, if the devitalized acellular matrix is intended for use on small wounds, micronization may be necessary prior to freeze drying.

Micronization of the tissue is accomplished by established methods including fracturing the acellular tissue after cryopreservation to obtain particulate material (step 26). Freeze drying is accomplished following established long standing industry methods (step 28). After micronization and subsequent freeze drying, the tissue may be given an additional ozone exposure to mildly crosslink the collagen, to increase the duration of time that the particulate acellular dermis will remain in the body before remodeling. The finished product material is then packaged and stored in a sterile material (step 30).

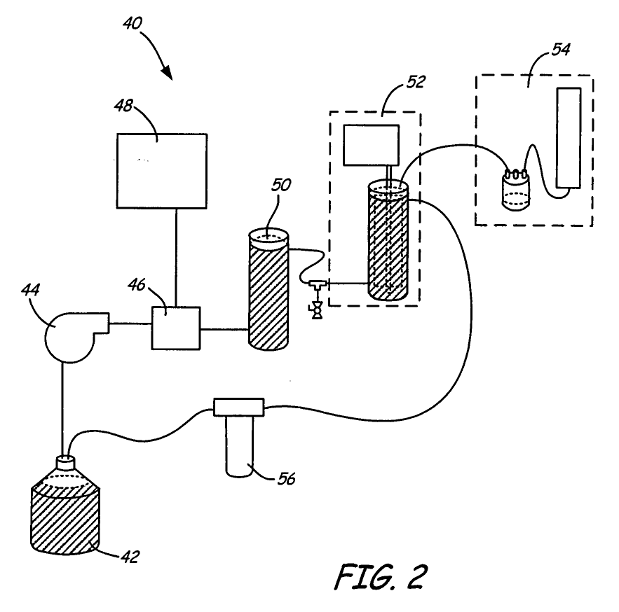

FIG. 2 is a diagram of system 40 for processing tissue to form a devitalized matrix for transplantation. System 40 includes fluid containment vessel 42, fluid pump 44, mixer 46, ozone generator 48, ozone concentration enhancement chamber 50, skin fixturing and processing chamber 52, fluid trap with ozone gas destructor 54, and fluid filtration unit 56. Balancing fluid flow rates and fluid retention times as well as proper skin exposure to the ozonated processing solution (for each component in system 40) is vital to its successful operation. All components of system 40 are specified with ozone compatibility and capable of full sterilization.

Fluid containment vessel 42 is utilized to prepare and transfer the processing fluid. Fluid containment vessel 42 may be formed of any suitable material, which may be properly cleaned and autoclaved/sterilized. One suitable material is stainless steel. In addition, fluid containment vessel 42 may also be formed of a polymeric material. Fluid containment vessel 42 is configured to have inlet and exit tubing and be of sufficient volume to maintain proper fluid flow volume throughout the entire system. As described above, pre-ozonated processing solution may consist of, but is not limited to, purified water, detergents, enzymes, surfactants, solvents, antimicrobials, penetrants, buffering agents and devitalization agents. This fluid is then pumped out of fluid containment vessel 42 by fluid pump 44 and into mixer 46 where it is infused with ozone gas.

Fluid pump 44 is used to pump processing solution through the multiple processing stages in of system 40, which are connected by suitable tubing attached to inlet and exit ports. In an exemplary embodiment, fluid pump 44 has a potential flow rate of about 0.5 to about 10.0 liters per minute. Adequate flow controls are required to obtain desired ozone concentration in solution, and to allow proper interaction and contact time between the mammalian skin product and the ozonated processing solution. Any suitable pump may be used. Peristaltic pumps have worked well because of flow control and sanitization reasons, but other pump types may also be utilized successfully.

Once the pre-ozonated processing solution is pumped from fluid containment vessel 42 into mixer 46 it is infused with ozone gas produced by ozone generator 48. Ozone generator 48 may be any commercially available unit that produces ozone gas in adequate volume and concentration for the devitalization of mammalian skin. Medical grade compressed oxygen may be used as the feed gas. Ozone flow rate is regulated by the flow rate of oxygen to ozone generator 48 and ozone gas output by ozone generator 48 is likewise controlled by the amount of ozone produced from the oxygen. In an exemplary embodiment, system 40 generates an oxygen flow rate of about 0.25 to about 5.0 liters per minute.

Mixer 46 provides for vigorous mixing and small ozone bubble formation to increase gas/fluid-surface interface, which increases the effective ozone concentration in the fluid. Ozone gas is injected into and blended with the processing solution in mixer 46. The ozone gas is then dissolved into the devitalized skin processing fluid in ozone concentration enhancement chamber 50 to form ozonated processing fluid.

It is desirable that system 40 produce a processing fluid with a high ozone concentration because increased ozone concentration relates to increased cell death and shorter skin devitalization processing times. Ozone concentration enhancement chamber 50 serves to increase ozone concentration before ozonated solution is pumped into skin fixturing and processing chamber 52. This is achieved by using commercially available equipment to inject ozone gas into the processing solution in a method consistent with increasing ozone concentration in the processing solution. In general, the amount of ozone gas in solution is maximized with a higher surface to volume ratio of gas to liquid, with lower temperatures and increased contact time between the ozone gas and liquid. Therefore, ozone is injected into the chamber at the bottom of ozone concentration enhancement chamber 50 utilizing mechanical diffusing units to vigorously mix the ozone gas and processing solution. This mixture is allowed an appropriate residence time in ozone concentration enhancement chamber 50 by regulating fluid flow through the chamber. Ozone concentrations are measured using a commercially available calorimetric test kit, such as those manufactured by The Hach Company of Loveland, Colo., on samples taken from a sample port installed in the tubing that connects ozone concentration enhancement chamber 50 and to skin fixturing and processing chamber 52. In an exemplary embodiment, the ozone concentration within the processing solution is about 0.5 to about 100-ppm, which has been shown to be effective in mammalian skin devitalization.

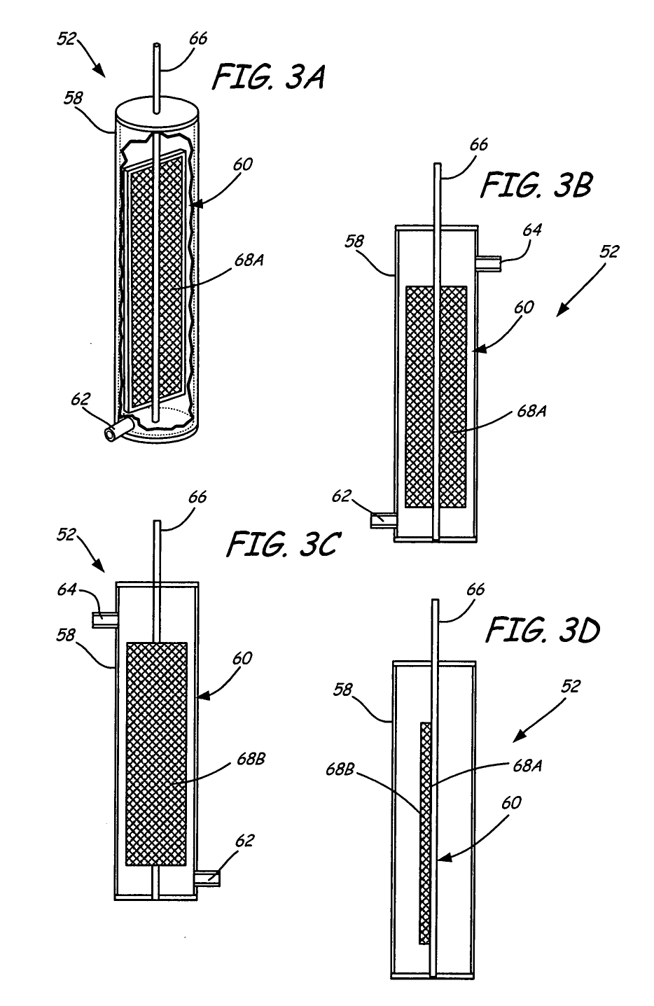

Skin fixturing and processing chamber 52 is a novel system for holding skin and maximizing skin exposure to the ozonated processing fluid. (Skin fixturing and processing apparatus 52 is described in detail in FIGS. 3A-3D.) Skin fixturing and processing apparatus 52 is comprised of a fluid containment vessel and a skin fixturing apparatus. The skin fixturing apparatus is configured to hold the tissue to allow for maximum exposure to the processing fluid. Ozonated processing fluid enters the bottom of skin fixturing and processing chamber 52 and flows over the skin before exiting out of a port positioned at the top of skin fixturing and processing chamber 52.

During the skin devitalization process, it is important to balance system 40 to maintain proper fluid flow and ozone gas into the fluid. Excess ozone gas released from the processing fluid can exit through the fluid trap and enter ozone destructor 54, where it is converted back to pure oxygen. It is important to control the release of excess ozone because ozone presents an inhalation hazard in sufficiently high concentrations.

Upon exiting skin fixturing and processing chamber 52, the processing fluid is pumped through tubing into fluid filtration unit 56. Fluid filtration unit 56 is configured to filter out tissue and other debris generated by the devitalization process. Single-stage or multiple-stage membranes may be employed to filter the processing solution. Filter surface area and pore size requirements are dictated by the surface area of skin processed and the level of desired debris removal. In an exemplary embodiment, a 2- to 20-square foot filter with a pore size ranging from about 1 to about 150 microns is used.

After passing through fluid filtration system 56, the processing fluid can be re-circulated through system 40 any desired number of times or the fluid can be discarded and fresh processing fluid introduced into the system.

FIGS. 3A-3D illustrate skin fixturing and processing chamber 52 in detail. Specifically, FIG. 3A is a perspective view of skin fixturing and processing chamber 52 and FIGS. 3B-3D are cross-sectional views of skin fixturing and processing chamber 52. Skin fixturing and processing chamber 52 is comprised of fluid containment vessel 58 and skin fixturing apparatus 60. Fluid containment vessel 58 includes first port 62 and second port 64. Skin fixturing apparatus 60 includes shaft 66 and first and second screens 68A, 68B, which are formed of any suitable mesh material.

As described above with respect to fluid containment vessel 42, fluid containment vessel 58 may be formed of any suitable material, which may be properly cleaned and autoclaved/sterilized, such as stainless steel or a polymeric material. First port 62 is positioned near the bottom of fluid containment vessel 58 and is used to connect fluid containment vessel 58 to ozone concentration enhancement chamber 50. Second port 64 is positioned near the top of fluid containment vessel 58 and is used to connect fluid containment vessel 58 to ozone destructor 54 and fluid filtration unit 56.

Skin fixturing apparatus 60 is positioned within fluid containment vessel 58 and configured to hold the tissue to allow for maximum exposure to the ozonated processing fluid contained within fluid containment vessel 58. Skin fixturing apparatus 60 includes shaft 66 which extends upward from the base of fluid containment vessel 58 and exits out of the top of fluid containment vessel 58. Shaft 66 is positioned centrally within fluid containment vessel 58 and supports first and second screens 68A, 68B, which are attached to shaft 66 such that first screen 68A is positioned parallel to second screen 68B. Shaft 66 is connected to a variable speed motor at its base that rotates shaft 66, and thus first and second screens 68A and 68B, at various desired speeds.

To restrain the skin during preparation and processing the skin is placed between first and second screens 68A, 68B such that the skin is adequately secured. This allows for maximum exposure to the ozonated processing fluid while fixing the skin in place. Skin fixturing apparatus 60 is then rotated as a desired speed within fluid containment vessel 58 to allow for increased fluid penetration into the skin and increased skin/ozonated-processing-fluid contact time.

### EXAMPLES

The present invention is more particularly described in the following examples that are intended as illustrations only, since numerous modifications and variations within the scope of the present invention will be apparent to those skilled in the art. Unless otherwise noted, all parts, percentages, and ratios reported in the following examples are on a weight basis, and all reagents used in the examples were obtained, or are available, from the chemical suppliers described below, from general chemical suppliers such as Sigma-Aldrich Company, Saint Louis, Mo., or may be synthesized by conventional techniques.

### Example 1

This example measured the effect of ozone on human skin. Human skin (epidermis and dermis intact) was exposed to ozone for various lengths of time. Ozone was pumped through an aqueous solution (sterile normal saline) obtaining levels between 5 and 20 ppm at temperatures between 4 C and 25 C. An alamarBlue™ viability assay, which is supplied by Invitrogen Corporation of Carlsbad, Calif., was used to quantify viability of the tissue cells. The alamarBlue™ dye is reduced by metabolic intermediates within a cell; reduction of the dye is then measured spectrophotometrically.

To perform the alamarBlue™ assay, human skin was sectioned into 6 mm diameter discs and placed individually into wells of a 24-well tissue culture plate. To each sample, 10% alamarBlue™ reagent in RPMI-1640 was added. The plate was incubated at 37° C. on an orbital shaker for 4 hours. Each well was then sampled in triplicate to a 96-well assay plate. The absorbance of the plate was measured at 600 nm and 570 nm, where the reduced and oxidized forms of the dye maximally absorb, respectively. The absorbencies were then used to calculate the percent reduction of alamarBlue™ dye, as follows:

$$\% reduced = \frac{( {ɛ}_{ox} {λ}_{2} ) ( A {λ}_{1} ) - ( {ɛ}_{ox} {λ}_{1} ) ( A {λ}_{2} )}{( {ɛ}_{red} {λ}_{1} ) ( {A}^{′} {λ}_{2} ) - ( {ɛ}_{red} {λ}_{2} ) ( {A}^{′} {λ}_{1} )} \times 100 ,$$

where:
ε_{red}λ₁=155,677, molar extinction coefficient of reduced alamarBlue™ at 570 nm
εₒₓλ₁=80,586, molar extinction coefficient of oxidized alamarBlue™ at 570 nm
ε_{red}λ₂=14,652, molar extinction coefficient of reduced alamarBlue™ at 600 nm
εₒₓλ₂=117,216, molar extinction coefficient of oxidized alamarBlue™ at 600 nm
Aλ₁=the absorbance of the test wells at 570 nm
Aλ₂=the absorbance of the test wells at 600 nm
A′λ₁=the absorbance at 570 nm of wells with alamarBlue™ and medium only
A′λ₂=the absorbance at 600 nm of wells with alamarBlue™ and medium only

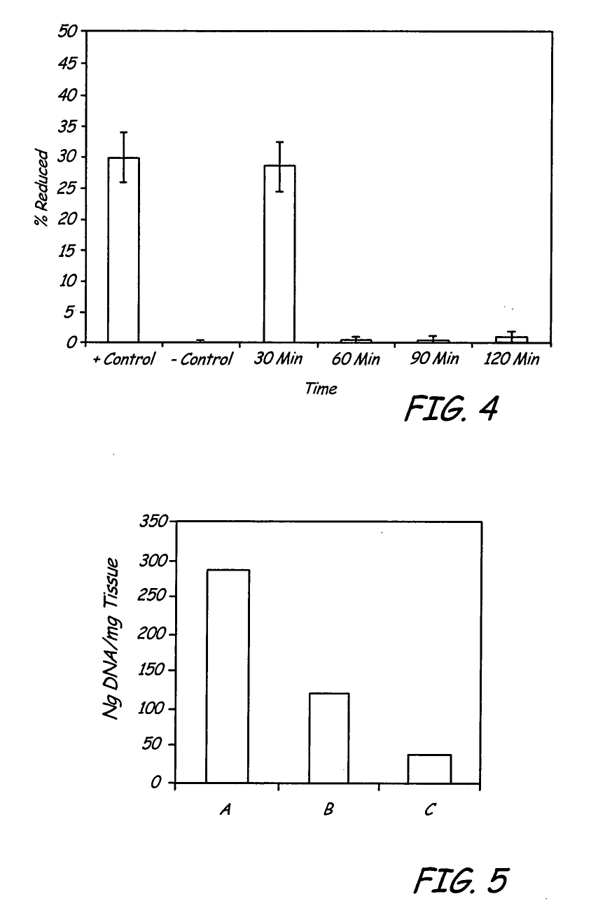

FIG. 4 is a chart which illustrates the results, which are presented as percent reduction, where greater reduction correlates to higher metabolic activity. As the chart shows, 60 minutes of ozone exposure in an aqueous solution (sterile normal saline) with agitation, elicited death of the cells within the skin.

### Example 2

This example demonstrates the antimicrobial ability of ozone delivered in an aqueous solution similar to treat tissue. S. epidermidis was subjected to 5-10 ppm ozone in sterile water, followed by plating the organisms onto agar plates and placing the plates in a 37° C. incubator. Table 1 below summarizes the results. As Table 1 shows, at 2.5 minutes or longer there was an 8 log reduction in colony forming units (CFU), representing total kill of microbes.

TABLE 1

| | S. epidermidis | | |
|---|---|---|---|
| Time Exposed | Initial Inoculum | Final | Log |
| to Ozone | (CFU/mL) | (CFU/mL) | Reduction |
| 2.5 min | 2.1 × 10⁸ | 0 | 8 |
| 5 min | 2.1 × 10⁸ | 0 | 8 |
| 7.5 min | 2.1 × 10⁸ | 0 | 8 |
| 10 min | 2.1 × 10⁸ | 0 | 8 |
| 20 min | 2.1 × 10⁸ | 0 | 8 |

### Example 3

This example illustrates the reduction in residual DNA using ozone treated devitalized human skin by subjecting it to a decellularization process and quantifying residual DNA after decellularization. Human skin was processed by devitalization with ozone, followed by removal of the epidermis by incubation in 1 M NaCl. Decellularization was performed by placing the tissue in a detergent wash using Tween 20 with subsequent rinses in sterile nanopure water, with endonucleases. The decellularization was performed in a chamber with agitation and flow-through solutions at 25° C. The DNeasy Blood & Tissue Kit manufactured by QIAGEN Inc. of Valencia, Calif. was used to purify DNA from tissue samples. Tissue samples were first sectioned into 25 mg pieces and cut into smaller pieces, followed by overnight proteinase K digestion at 56° C. Once the samples were fully lysed, the samples were added to a DNeasy Mini spin column and centrifuged to bind DNA to the spin column membrane. DNA binding was followed by wash steps using supplied buffers to remove any contaminants. Purified DNA was collected by eluting the DNA from the cleaned membrane by centrifugation with elution buffer. The extracted DNA was then quantified with a spectrophotometer.

FIG. 5 illustrates the results of this study. Results are reported as DNA concentration (ng/μl). The amount of total DNA per weight of tissue was calculated by the following equation:

(DNA(ng/μL)×elution volume(μL))/sample weight(mg)=ng DNA/mg tissue

As shown in FIG. 5, there was a greater than 90% reduction in DNA content following decellularization of the dermal matrix.

Therefore, these examples demonstrate that the novel application of ozone to tissue as a devitalizing agent kills living cells inhabiting the tissue, as shown in Example 1. Exposing microorganisms to ozone in a solution results in significant kill of the bacteria, showing the potential for ozone as an agent for bioburden reduction and decontamination, demonstrated in Example 2. Finally, ozone treated devitalized tissue may undergo a decellularization process as shown in Example 3, with a reduction of residual DNA of greater than 90%, thereby creating a tissue matrix with minimal residual antigenic material.

Although the present invention has been described with reference to preferred embodiments, workers skilled in the art will recognize that changes may be made in form and detail without departing from the spirit and scope of the invention.

1. A method for processing tissue to produce a devitalized acellular matrix for transplantation, the method comprising:
soaking the tissue in a first processing solution having a pH below 7 to reduce protease activity;
periodically infusing ozone into the first processing solution to devitalize the tissue and reduce bioburden; and
soaking the tissue in a second processing solution to remove cellular debris.

2. The method of claim 1, and further comprising:
stabilizing the tissue in an isotonic nutrient storage media at 4° C. prior to soaking the tissue in the first processing solution.

3. The method of claim 1, and further comprising:
cryopreserving the tissue prior to soaking the tissue in the first processing solution.

4. The method of claim 3, and further comprising:
thawing the cryopreserved tissue and soaking it in the first processing solution, followed by soaking the tissue in the second processing solution.

5. The method of claim 1, and further comprising:
exposing the tissue to ozone after soaking the tissue in the second processing solution to further reduce bioburden and crosslink the tissue.

6. The method of claim 1, and further comprising:
cryopreserving the tissue after soaking the tissue in the second processing solution.

7. The method of claim 6, and further comprising:
packaging the tissue in sterile, vapor-permeable material.

8. The method of claim 6, and further comprising:
micronizing the tissue following cryopreservation.

9. The method of claim 8, and further comprising:
exposing the tissue to ozone after micronization to crosslink the tissue.

10. The method of claim 1, and further comprising:
agitating the tissue while soaking in the second processing solution to remove debris, followed by a placing the tissue in a rinsing solution to thoroughly remove any residual processing solutions.

11. The method of claim 1, and further comprising:
packaging and freeze drying the tissue after soaking the tissue in the second processing solution.

12. The method of claim 11, and further comprising:
micronizing the tissue prior to freeze drying.

13. The method of claim 11, and further comprising:
packaging the tissue in sterile vapor-permeable material.

14. The method of claim 1, wherein the first processing solution contains protease inhibitors.

15. The method of claim 1, wherein the first processing solution has a pH of about 5.0 to about 6.8.

16. The method of claim 1, wherein ozone is infused into the first processing solution for a time period ranging from about 1 minute to about 3 hours.

17. The method of claim 16, wherein a concentration of ozone in the first processing solution is about 0.5 ppm to about 100 ppm.

18. The method of claim 1, wherein ozone is infused into the second processing solution for a time period ranging from about 1 minute to about 3 hours.

19. The method of claim 1, wherein the second processing solution is comprised of detergents and endonucleases.

20. The method of claim 1, wherein the tissue is soft tissue.

21. The method of claim 20, wherein the soft tissue is selected from the group consisting of skin, blood vessel, nerve, muscle, tendon, pericardium, dura, fascia lata, placenta, omentum tissue and combinations thereof.

22. The method of claim 20, and further comprising:
separating a dermis portion of the skin from an epidermis portion of the skin after soaking the skin in the first processing solution.

23. The method of claim 22, wherein the dermis portion of the skin is soaked in the second processing solution.

24. The method of claim 1, wherein the tissue is mammalian skin tissue.

25. A system for processing tissue to form a devitalized acellular matrix for transplantation, the system comprising:
a first fluid containment vessel for containing a processing fluid;
an ozone generator for producing ozone gas;
a fluid pump connected to the first fluid containment vessel;
a mixing system connected to the fluid pump and the ozone generator for infusing the processing fluid with ozone to produce an ozonated processing solution;
an ozone concentration chamber for receiving the ozonated processing fluid and increasing ozone concentration;
a second fluid containment vessel connected to the ozone concentration chamber, wherein the second fluid containment vessel receives the ozonated processing fluid;
a tissue fixing apparatus disposed within the second fluid containment vessel to hold a piece of tissue;
a fluid filtration unit connecting to the second fluid containment vessel and the first fluid containment vessel, wherein the fluid filtration unit receives processing fluid from the second fluid containment vessel and returns the processing fluid to the first processing vessel after filtration; and
a fluid trap comprising an ozone destructor in connection with the second fluid containment vessel, wherein excess ozone gas released from the processing fluid can exit through the fluid trap and enter the ozone destructor where it is converted back to pure oxygen.

26. The system of claim 25, wherein the ozone generator produces ozone at a rate of about 0.25 liters per minute to about 10.0 liters per minute.

27. The system of claim 25, wherein the fluid pump has a flow rate of about 0.5 liters per minute to about 10.0 liters per minute.

28. The system of claim 25, wherein the tissue fixing apparatus comprises a first screen and a second screen disposed opposite the first screen.

29. The system of claim 29, wherein the tissue may be held between the first screen and the second screen.

30. The system of claim 29, wherein the tissue fixing apparatus further comprises a shaft having a proximal end and a distal end in which the proximal end is in connection with the first and second screens and the distal end is in connection with the bottom of the second fluid containment vessel.

31. The system of claim 25, wherein the tissue fixing apparatus is rotatable with respect to the second fluid containment vessel.

32. The system of claim 25, wherein the tissue fixing apparatus is connected to a motor which rotates the tissue fixing apparatus.
